(19) United States
(12) Patent Application Publication
(10) Pub. No.: US 2023/0142937 A1
(43) Pub. Date: May 11, 2023

(54) ELECTRONIC STETHOSCOPE

(71) Applicants: RUPAK KUMAR JHA, Singapore (SG); Meena Kumari Selvarengaraju, Singapore (SG)

(72) Inventors: RUPAK KUMAR JHA, Singapore (SG); Meena Kumari Selvarengaraju, Singapore (SG)

(21) Appl. No.: 17/523,397

(22) Filed: Nov. 10, 2021

(51) Int. Cl. A61B 7/04; A61B 5/00

(52) U.S. Cl. CPC A61B 7/04; A61B 5/7475; A61B 5/002; A61B 5/0022; A61B 5/6843; A61B 5/742; A61B 2562/0204; A61B 2560/0247; A61B 2562/0247

(57) ABSTRACT

An electronic stethoscope uses a contact sensor to confirm continuous contact with a patient. A recording of body sounds is begun while a timer is initiated. Upon a signal from the timer, recording is terminated. The duration of the timer may be set remotely by a practitioner. Additionally, the contact sensor may require a minimum level of force as an implicit indication that the stethoscope is firmly stationary. Once a valid measurement is recorded, it is analyzed in comparison to a baseline. Artificial intelligence is used to select the appropriate from a database of sampled data. Bootstrapping is used to develop additional data sets and Random Forest algorithm is used to select the appropriate baseline from the data. The current recording is analyzed with respect to the AI selected baseline. A display on the electronic stethoscope displays analyzed results as well as providing the visual aspect of a user interface.

### FIELD OF THE INVENTION

This invention relates to electronic stethoscopes. More specifically, this invention relates to electronic stethoscopes that apply artificial intelligence to compare and analyze readings and which have wireless capabilities allowing remote practice of medicine, or telemedicine.

### BACKGROUND OF THE INVENTION

The stethoscope is a medical device used by doctors, nurses, and other healthcare professionals to listen to sounds from human or animal body. Health care professionals use stethoscopes to listen to the sounds from heart, lungs, arteries and veins, intestines, mother's womb to diagnose based on the sounds received. After well over a century of use, the stethoscope is a ubiquitous diagnostic tool and practitioners have considerable training, experience, and comfort with a stethoscope.

The conventional widely used stethoscope consists of chest piece, flexible rubber tube, and earpiece all connected. Typically, the chest piece itself has two surfaces that may be applied to a patient for auscultation. Theses surfaces are the diaphragm and the bell. The diaphragm is a plastic or fiber glass disc fixed tightly to a circular rim. Behind the diaphragm is a chamber with a conical back opposite the diaphragm to direct sound to an aperture. The bell is a shallow open cup with an aperture in it. The cup shape of the bell directs sounds to the aperture. The apertures from the diaphragm and the bell lead to passages that lead to the flexible rubber tube. The flexible rubber tube in turn conducts sound to the earpiece(s). The bell transmits low frequency sounds, whereas the diaphragm transmits high frequency sounds. When the diaphragm of a stethoscope is placed on the human or animal body, the diaphragm vibrates according to body sounds, creating acoustics pressure waves which are directed to the aperture and travel up the tube to the earpiece. Health care professionals places the earpiece on their ears to listen to the sound from diaphragm.

With constantly improving electronics and communication technology, the field of telemedicine has consistently expanded in its reach and in its fields of application. With mobile communication technology, less hard infrastructure is needed for communicating over great distances, so that patients in remote locations may still have access to practitioners. With the miniaturization of electronics and the improvement of data transmission, digitized information can be gathered in remote locations and transmitted for analysis or stored. All of this was expanding the reach of telemedicine. Additionally, while it is natural to think of the patient as a person, the patient could be an animal. Telemedicine has extended the reach of species specialists, and as a result, telemedicine has expanded among veterinarians as well. Therefore, when the terms patient or body are used, the reference may also be to an animal.

In 2019-2020, the globe was hit by a pandemic commonly called COVID-19, also known as Coronavirus pandemic. This pandemic was caused by severe acute respiratory syndrome coronavirus 2 (SARS-CoV-2). Virus spreads though air when infected person nearby cough, breathe, or sneeze. It may also spread when contaminated surfaces are touched. In general, travel and personal interaction were greatly reduced during the pandemic. Additionally, due to concern about disease spread by physical contact, doctors and nurses faced difficulty carrying the stethoscope around, sterilizing it, and using it with the Personal Protection Equipment (PPE) kit worn by them. Due to the pandemic, there was a continued and expanding need for remote doctor consultation (Tele-consultation), which spurred the application of telemedicine even further. Tele-consultation and telemedicine still require the ability to collect diagnostic information such as provided by a traditional stethoscope.

Although electronic stethoscopes provide advantages, there are problems associated with them as well. The heighted sensitivity of electronic stethoscopes can introduce noise problems which must be addressed if the electronic stethoscope is to be effective. In many environments, ambient noises are significant. Another source of noise is located at the stethoscope itself. If the diaphragm face of the stethoscope is moving with respect to the surface it is contacting, clothing or skin, a significant noise component is generated. Both of these noise components should be addressed to gain the benefits of an electronic stethoscope.

When an electronic stethoscope is used for remote medical consultations, the user of the electronic stethoscope may not be a practitioner. To insure acquisition of good signals and measurements, it is desirable that a remote practitioner has some control over the acquisition of a measurement. One factor associated with this is the duration of the measurement wherein a minimal length of time for a measurement may establish a higher quality measure and provide better diagnostic information. Additionally, diagnoses often entail comparison to baselines. Information associated with a given patient captured along with the measurement provides a better selection of the baseline.

### SUMMARY OF EMBODIMENTS OF THE INVENTION

An electronic stethoscope has a housing with a diaphragm at a surface of the housing and electronics within the housing. The electronics of the electronic stethoscope comprise: a sound sensor; a programmable chip; a battery; a contact sensor; a display; user interface controls; a port; wireless communication elements; and other electronic elements. The electronics are distributed variously about the electronic stethoscope on circuit boards, etc. with some visible and accessible to users.

A chest piece is positioned within the housing behind the diaphragm, and the sound sensor is positioned to receive and record sound transmitted by the diaphragm and chest piece. The sound sensor comprises a transducer and processor for filtering, conditioning, and converting from analog to digital signals. In some embodiments, the sound sensor comprises a microelectromechanical system (MEMS) sensor combining transducer and conditioning functions and some embodiments may comprise an additional sensor chip for conditioning and transmission functions. In still other embodiments, the sound sensor may comprise a piezo-electric sensor.

The sound sensor transmits a signal to the programmable chip for storage, analysis, additional processing, transmission to other elements. In some embodiments, programmable chip itself has wireless communication capabilities and can transmit the signal received from the sound sensor. Programmable chip executes machine readable instructions to analyze the signal from the sound sensor, drive the display, receive signals from a user interface, receive signals from the contact sensor, and in general operate and coordinate the other elements of the electronic stethoscope. The machine readable instructions for the programmable chip may modified via wireless communications or the port which also provides a means for recharging the battery.

The contact sensor is located proximal to the rim. The contact sensor detects when the rim is in contact with a body and sends a signal to the programmable chip. With confirmation of contact between the diaphragm and a body, the programmable chip initiates a timer while receiving and recording signals from the sound sensor. The duration of the timer may be adjusted by the user on location or the duration may be remotely adjusted by a consultant. The electronic stethoscope may provide cues such as audible cues to indicate when a timer has been initiated, and when the electronic stethoscope may be moved. In this way, it is assured that a sound sample is of sufficient length for diagnostic purposes.

In some embodiments of the electronic stethoscope, the signal from the contact sensor may prompt other steps by the programmable chip. Motion of the diaphragm along a surface such as skin or clothing can generate a surge of noise. For contact sensors capable of measuring force, a minimum threshold of force is interpreted by the programmable chip as indicating that the electronic stethoscope is firmly in place and stationary. With the stethoscope in place, signals from the sound sensor can be recorded and processed without a surge of noise into the signal and resulting audio file.

### BRIEF DESCRIPTION OF THE DRAWINGS

Additional utility and features of the invention will become more fully apparent to those skilled in the art by reference to the following drawings, which illustrate some of the primary features of preferred embodiments.

FIG. 1 is a side perspective view of an embodiment of an electronic stethoscope.

FIG. 2 is a perspective view of the listening end of an embodiment of an electronic stethoscope.

FIG. 3 is a perspective view of an embodiment of a chest piece in an embodiment of an electronic stethoscope.

FIG. 4 is a cross-sectional view of an embodiment of an electronic stethoscope.

FIG. 5 is a top view of a first peripheral circuit board.

FIG. 6 is a top view of a second peripheral circuit board.

FIG. 7 is a top view of a central circuit board of an embodiment of an electronic stethoscope.

FIG. 8 is a top view of a sensor ring to fit around the diaphragm of an electronic stethoscope.

FIG. 9 shows a strain gauge.

FIG. 10 is a perspective view of a force sensitive resistor (FSR).

FIG. 11 is a flow chart of recording, signal processing, and communication of a measurement.

FIG. 12 shows interaction between elements of the system.

FIG. 13 shows the timed recording of a measurement through signal processing to automated analysis and comparison of the measurement.

FIG. 14 is an example of a table, Table 1, containing a sampled data set.

FIG. 15 is an example of a table, Table 2, containing a Bootstrap data set constructed from Table 1 of FIG. 14.

FIGS. 16 and 17 show two decision trees created over two variables in reverse order.

### DETAILED DESCRIPTION OF EMBODIMENTS

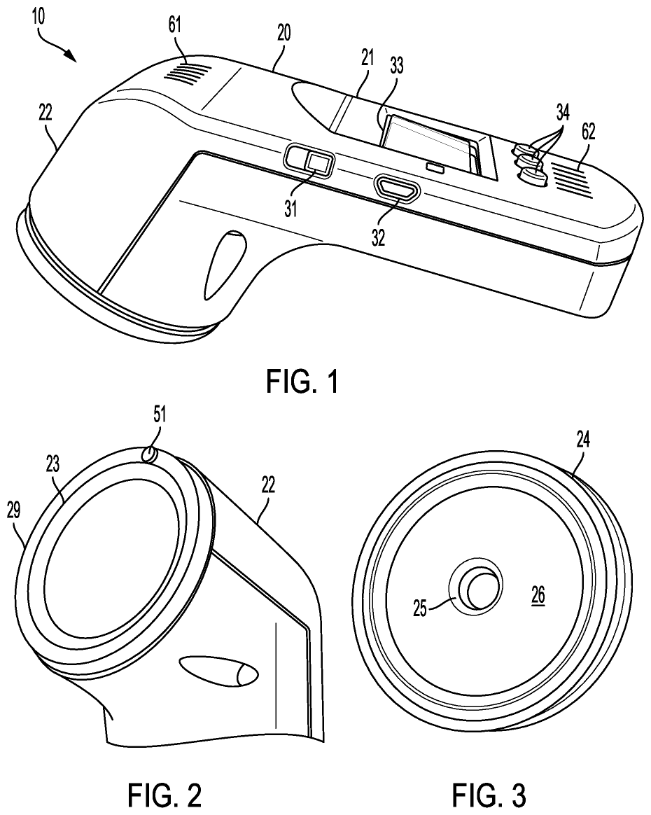

FIG. 1 is a side perspective view of an embodiment of an electronic stethoscope 10. Housing 20 of electronic stethoscope 10 houses electronics of electronic stethoscope 10 and has multiple apertures for user controls, measurements, and communications. Power switch 31 on handle 21 of electronic stethoscope 10 provides manual control of the state of electronic stethoscope 10. Port 32 on handle 21 of electronic stethoscope 10 provides a connection for charging electronic stethoscope 10. The configuration of port 32 is not limited to that shown in FIG. 1. Any desirable port may be employed. Port 32 also provides another means of input and output for electronic stethoscope 10. Information may be downloaded via port 32, and port 32 may also be used to upload updates of information and firmware to electronic stethoscope 10.

Display screen 33 and control buttons 34 on handle 21 of electronic stethoscope 10 provide a user interface with electronic stethoscope 10. Display screen 33 may be an LED display or any suitable display. In one embodiment of an electronic stethoscope 10, control buttons 34 comprise three buttons for navigating through options shown on display screen 33 to control electronic stethoscope 10. Any suitable interface may be used for control buttons 34. In some embodiments of electronic stethoscope 10, control buttons 34 may be micro-switch push buttons or any similar suitable push button. Other embodiments of electronic stethoscope 10 may employ capacitive touch sensor buttons to navigate through options on display screen 33. Still other embodiments may employ touch sensitive screens. Control buttons 34 may comprise a button each for “Up”, “Down”, and “Select”. These allow a user to move through options displayed on display screen 33 and select choices in decision trees or select functions to operate electronic stethoscope 10. Additionally, the number of input buttons may be changed as desired.

Head 22 on electronic stethoscope 10 houses some of the audio components of electronic stethoscope 10. In some embodiments of electronic stethoscope 10, head 22 may have ambient apertures 61 to allow passage of sound between the interior and exterior of housing 20. In some embodiments of electronic stethoscope 10, handle 21 may have sound apertures 62 to allow passage of sound between the interior and exterior of housing 20.

FIG. 2 is a perspective view of head 22 of an embodiment of electronic stethoscope 10. Diaphragm 23, acting as a surface of housing 20, covers end of head 22, and helps to “pick up” higher and lower frequency body sounds when head 22 of electronic stethoscope 10 is applied to a body. Diaphragm 23 may be made of any suitable material such as Bakelite. Head 22 contains a chest piece similar to those of standard stethoscopes. The chest piece is positioned behind diaphragm 23 to direct and transmit sounds received from diaphragm 23. Rim 29 holds diaphragm 23 in place.

FIG. 3 is a perspective view of an embodiment of chest piece 24 for directing sound received from diaphragm 23. Chest piece 24 is frequently made of metal, such as aluminum, zinc alloy, etc. Aperture 25 through chest piece 24 allows sound to pass through chest piece 24. A sound sensor transduces the sound transmitted by diaphragm 23 and directed by chest piece 24 into an electric signal and transmits it to other electronic elements of electronic stethoscope 10. The sound sensor may be comprised of multiple elements, such as a transducer, signal conditioner, analog-to-digital (AD) converter, signal filter, and interface.

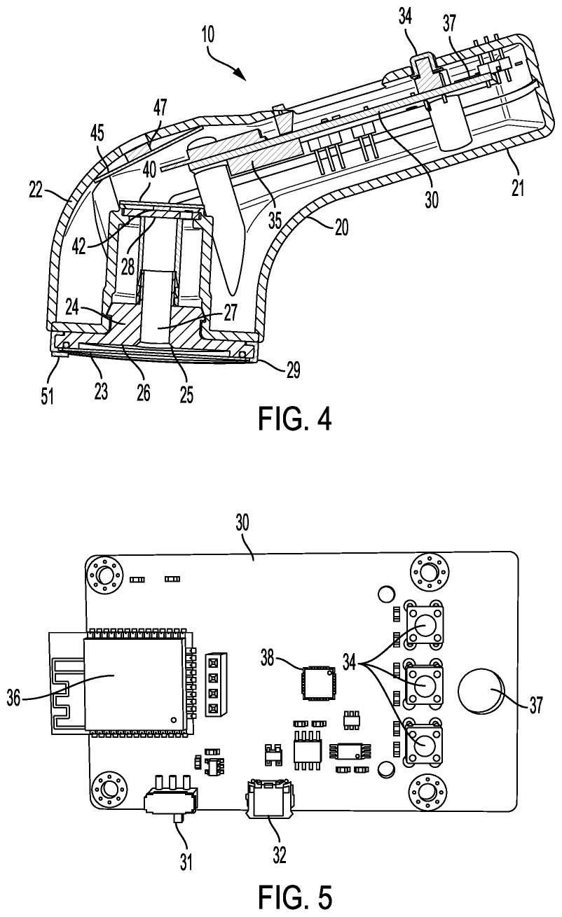

FIG. 4 is a cross-sectional view of an embodiment of electronic stethoscope 10. In the embodiment shown in FIG. 4, head 22 is configured to reproduce the auditory characteristics of standard stethoscopes. This facilitates the use by practitioners having extensive experience with standard stethoscopes. Other embodiments may not reproduce the performance of standard stethoscopes as closely. Head 22 houses chest piece 24 which is covered by diaphragm 23. Rim 29 holds diaphragm 23 to chest piece 24. Chest piece 24 has an aperture 25 centrally located in its face 26. Audio tube 27 extends from aperture 25 back into head 22 where it terminates. First peripheral circuit board 40 at the end of audio tube 27 carries sound sensor 41. Sound sensor 41 records sounds from audio tube 27. When diaphragm 23 is placed on a body, diaphragm 23 vibrates to any sounds produced from the body and creates sounds waves that are transmitted into chest piece 24. These sound waves are guided to aperture 25 by the shape of face 26 on chest piece 24. Tube 27 guides the sound waves back to sound sensor 41 on first peripheral circuit board 40 at the internal end 28 of tube 27.

FIG. 5 is a top view of first peripheral circuit board 40. In the embodiment shown in FIG. 5, sound sensor 41 comprises multiple components, sound transducer 42 and sensor chip 43. Sound transducer 42 records the sound waves, converts them to an electrical signal, and transmits the resultant signals to sensor chip 43 of sound sensor 41, which performs signal conditioning, conversion of the signal from analog to digital, anti-aliasing filtering, and feeds the signal to an I2S interface for communication to a central processor for analysis, external transmission, etc. Transducer 42 may be a MEMS (microelectromechanical system) sensor, or any other suitable sound transducer, and may itself have filtering and conditioning capabilities.

Returning to FIG. 4, sound sensor 41 on first peripheral circuit board 40 is situated to record sounds conducted to it via audio tube 27. To a great extent these will be sounds produced from within the body being monitored by electronic stethoscope 10. However, to some degree ambient sounds from the surrounding environment can also be transmitted via the body and therefore be picked up by sound sensor 41. The physical structure of electronic stethoscope 10 may also conduct these ambient sounds. For the purposes of electronic stethoscope 10 these ambient sounds are noise, and it is desirable to reduce their appearance in the signal produced by sound sensor 41. In some embodiments of electronic stethoscope 10, a second sound sensor, or microphone is used to directly sample ambient noise.

Microphone 46 at the back of head 22 of electronic stethoscope 10 is oriented to record ambient sounds, or noise. Microphone 46 may record ambient noise transmitted through housing 20, or in some embodiments, microphone 46 is directly exposed to the ambient environment via ambient apertures 61 (see FIG. 1). Microphone 46 is mounted on second peripheral circuit board 45 along with any peripheral chips for signal conditioning and processing. Microphone 46 may itself be a MEMS (microelectromechanical system) sensor or any other suitable sound transducer, and may itself have filtering and conditioning capabilities. Microphone 46 records ambient sounds, and the signal resulting from the ambient sounds is inverted and passed to sound sensor 41 to cancel the ambient sound component in the signal from sound transducer 42. This results in a signal representing only the body sounds detected by sound transducer 42. This signal is transmitted to a central processor.

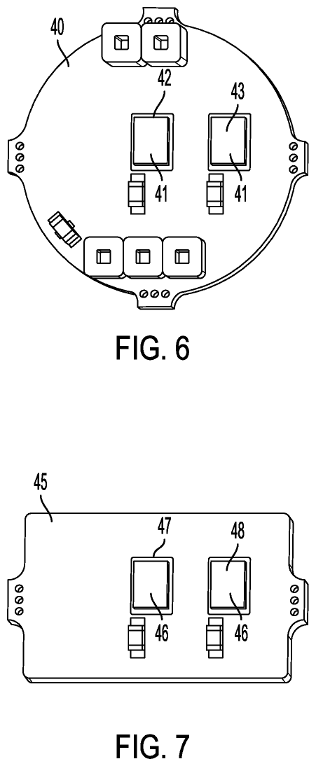

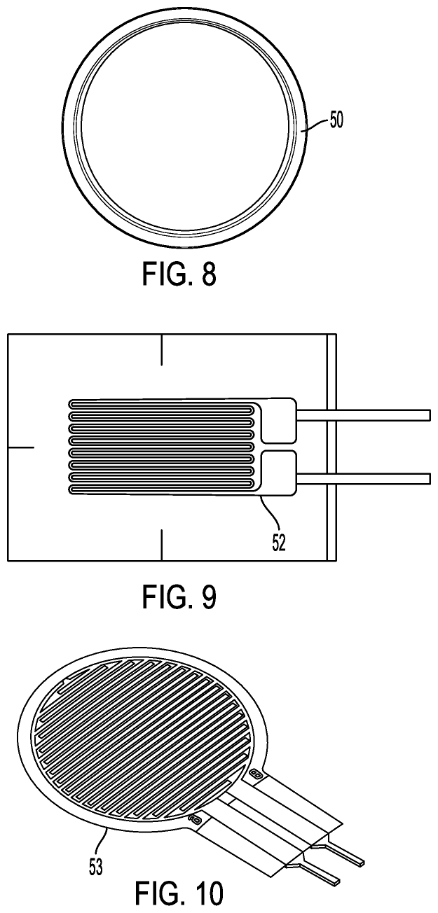

FIG. 6 is a top view of a second peripheral circuit board 45. In the embodiment shown in FIG. 6, sound sensor 46 comprises a MEMS (microelectromechanical system) sensor as transducer 47 and sensor chip 48. These elements record ambient sounds, invert the resulting electrical signal and transmit the inverted signal to sound sensor 41 to be used to cancel ambient components of the signal at sound sensor 41.

FIG. 4 shows a cross-section of central circuit board 30 with battery 35 mounted on the bottom side of central circuit board 30. Battery 35 is chargeable through port 32. FIG. 5 is a top view of central circuit board 30 of an embodiment of electronic stethoscope 10. Previously discussed power switch 31, port 32, and control buttons 34 are mounted on central circuit board 30. Programmable chip 36 executes machine instructions to interact with and control other elements of electronic stethoscope 10 and apply algorithms to process and analyze the signal received from sensor chip 43. Sensor chip 43 is capable of applying different frequency filters to different body sound measurements. For example, a different filter may be applied to heart sounds than is applied to lung sounds. Inputs into programmable chip 36 may result in sensor chip 43 applying different processing and filters to the signal from sound transducer 42. In the embodiment of central circuit board 30 shown in FIG. 5, programmable chip 36 is also capable of wireless transmission and receiving of signals in at least Bluetooth and Wi-Fi modes, including in encrypted formats. With wireless transmission, electronic stethoscope 10 is able to communicate with external devices and to interact with Cloud applications. Electronic stethoscope 10 may also receive firmware updates wirelessly and have some settings controlled and adjusted remotely. Remote adjustments allow a practitioner to change settings of the stethoscope remotely while a lay person on site applies electronic stethoscope 10 to a body.

Some embodiments of electronic stethoscope 10 may produce audible cues while it is in operation. Sound generator 37 on central circuit board 30 produces sounds and is driven by programmable chip 36 executing machine readable instructions. Sound generator 37 may generate audible cues to indicate: beginning of recording; ending of recordings; error conditions; etc. In some embodiments of electronic stethoscope 10, sound apertures in housing 20, such as sound apertures 62 in FIG. 1, may facilitate the emission of sounds from sound generator 37.

In some applications, it is desirable to control the duration of the measurement recorded by electronic stethoscope 10. For example, whether the heart or lungs of a patient are being monitored may determine the desired time duration of a measurement. For a valid reading, or recording, of body sounds, diaphragm 23 must be in sufficient contact with the body for the duration of the reading. When the reading duration is being automatically timed, a contact sensor needs to be employed to monitor the contact of diaphragm 23 with the body. In FIGS. 3 and 4, at least the measuring, or transducer, part of contact sensor 51 is located proximal to diaphragm 23. Other elements of contact sensor 51, such as its control chip, etc. may be located elsewhere, such as on central circuit board 30. The measurement commences when the contact sensor indicates diaphragm 23 is in contact with a body and diaphragm 23 is in position. If electronic stethoscope 10 is left in contact longer than needed, timers in electronic stethoscope 10 terminate measurement. Embodiments of electronic stethoscope 10 having audible sound generators, such as sound generator 37, may provide audible cues as to when diaphragm 23 is in position, and the measurement has begun. When enough time has elapsed that head 22 may be removed, an additional cue may be emitted. The duration of the measurement may be remotely set by a practitioner via the wireless communications.

A variety of contact sensors may be employed for the purpose of monitoring contact between diaphragm 23 and a body. This allows the duration and quality of the measurement to be controlled. The contact sensor may be comprised of multiple elements. FIG. 8 is a top view of sensor ring 50 which fits around diaphragm 23 of an embodiment of electronic stethoscope 10. FIG. 9 shows strain gauge 52. FIG. 10 is a perspective view of force sensitive resistor (FSR) 53. These elements, or combinations of these elements, and their respective electronic control chips combine to operate as contact sensors.

In some embodiments of electronic stethoscope 10, electronic sensors may be employed to incorporate sensor ring 50. These sensors monitor electromagnetic effects at sensor ring 50. For example, capacitive sensors use the phenomenon of capacitance to detect when sensor ring 50 is being touched. Other sensors may measure conductivity across the face of sensor ring 50 to detect when sensor ring 50 is touching a body.

Some contact sensors may monitor sensor ring 50 for mechanical measurements. These sensors detect when a force is applied to sensor ring 50, or they detect when sensor ring moves with respect to head 22. With mechanical sensors that detect when sensor ring 50 moves, sensor ring 50 is mounted in such a fashion that it can move a finite distance so that a switch monitoring sensor ring 50 can detect the motion. A biasing element such as a spring or elastic boot biases sensor ring 50 to an initial position. Placing head 22 in contact with a body moves sensor ring 50 and this movement is detected. Any applicable sensors may be employed for detecting the movement of sensor ring 50. These may include binary switches that have their state changed by movement of sensor ring 50. In other embodiments proximity sensors may be used to detect movement of sensor ring 50. In those case, sensor ring 50 has at least a portion that is metal. In other applications, sensor ring 50 may itself close contacts present on head 22. Again, in those embodiments, sensor ring 50 has portions that are metallic.

Mechanical sensors that measure force to detect when sensor ring 50 is placed against a body need less motion from sensor ring 50, and in some embodiments may not require the presence of sensor ring 50. In some embodiments, the force is measured at a biasing element exhibiting strain due to force at sensor ring 50. Strain gauge 52 is incorporated into a Wheatstone bridge in standard application of a strain gauge to measure force through the strain exhibited at a biasing element associated with sensor ring 50. Alternatively, force sensitive resistor (FSR) 53 can provide a more direct measurement of force. Force sensitive resistor 53 varies it resistance based on the force applied, and this is measured by accompanying electronics. In some embodiments, FSR 53 may measure the force directly between a location on electronic stethoscope 10 and a body. For example, FSR 53 may be located on, or near, diaphragm 23. The electronics within electronic stethoscope 10 can be set to interpret a given threshold force measured by strain gauge 52 or FSR 53 as indicating that sensor ring 50 is pressing against a body. If the force falls below the programmed threshold, it is interpreted as sensor ring 50 ceasing to contact a body.

Once the electronics of electronic stethoscope 10 detect that head 22 is placed against a body, the electronics begin capturing the signals from sensor chip 43. Electronic stethoscope 10 continues recording the measurement until the time terminates. Electronic stethoscope 10 may emit cues to communicate that electronic stethoscope 10 may be removed from contact with the body. These cues may be audible cues from a source such as sound generator 37 or visual cues from display screen 33 or other visible source.

The electronics of electronic stethoscope 10, such as programmable chip 36, may be programmed to perform several real-time operations with the signal. These operations include processing, encapsulating, analyzing, comparing, and transmission of the signal to external devices and networks. The firmware of electronic stethoscope 10 is updatable and can accommodate multiple transmission formats such as Bluetooth, Wi-Fi, 4G. 5G, etc. Electronic stethoscope 10 and the larger system may also store the signal for later analysis. This storage may also be Cloud-based.

Some embodiments of electronic stethoscope 10 may make further use of a contact sensor that detects when diaphragm 23 is in stationary contact with a body. If diaphragm 23 is rubbed, or moved, along a surface such as skin or clothing, a surge of noise is generated. Due to the need for high gain to record body sounds, this motion generated noise can be quite loud at the auditory output to the point of being unpleasant, even painful, to a listener.

Embodiments of electronic stethoscope 10 that employ a contact sensor that registers force at diaphragm 23, or head 22 more generally, can use the force detection as a proxy signal regarding motion. A designated minimum level of force registered at diaphragm 23 is evaluated as indicating that head 22 is stationary as it is being pressed against a body. With sufficient force registered, programmable chip 36 records and processes the signal from sound sensor 41. If the force indicated by the contact sensor is below a predetermined, or preset, threshold, digital auditory signals received by programmable chip 36 are attenuated to avoid auditory “spikes” in recorded files and a file may not even be recorded and saved. Insufficient force measured at diaphragm 23 is interpreted as indicating that electronic stethoscope 10 is not established at a stationary position on a body.

Referring back to FIG. 4, contact sensor 51 may be a force sensitive resistor (FSR) 53 positioned on the face of head 22. FSR 53 is capable of registering forces over a range, and the firmware of electronic stethoscope 10 can be adjusted to require a minimum force at FSR 53 to record a signal received at programmable chip 36 from sound sensor 41. Programmable chip 36 may signal via other elements of electronic stethoscope 10, such as light emitting diodes, display screen 33, sound generators, etc., that an operator is holding electronic stethoscope 10 firmly enough to a body.

Although FSR 53 provides a contact sensor 51 that can detect force continuously over a range, a binary signal arrangement may also be employed. For example, any embodiment employing a spring biased sensor ring 50, could be configured to require a minimum compression which then correlates to minimum force at head 22. A binary switch can detect the minimum compression, i.e. minimum force, and signal to programmable chip 36 which interprets the signal as an indication that electronic stethoscope 10 is in place. Additionally, a binary switch may be calibrated to detect a minimum force directly between electronic stethoscope 10 and a body.

Electronic stethoscope 10 is capable of capturing and recording other information associated with a file of body sounds. Various embodiments of electronic stethoscope 10 may capture the geolocation, altitude, ambient temperature, humidity, pollen count, pollution readings, patient temperature, contemporaneous blood oxygen level, blood pressure, and other factors. Some of this information may be detectable directly by electronic stethoscope 10, and some of this information may be entered with the user interface for electronic stethoscope 10. Accessories may also be used to enter this supplementary information such as via port 32 or via wireless communications. This information may be incorporated into the data file encapsulating the digital file of body sounds, and provides context for interpreting the electronic stethoscope 10 reading of body sounds in specific cases. The environment in which a reading is taken may itself be a stressor. For example, high heat and humidity may cause distress to a patient, and readings from high altitudes would have different characteristics. More generally, the additional information accompanying readings provide long term data for locations and environmental effects, both for individual histories and for populations and locales.

Electronic stethoscope 10 may also accommodate entry of individual profile information such as age, sex, ethnicity, etc. Display screen 33 and control buttons 34 may be used to navigate questionnaires for simple answers. More complicated answers and background information may be captured via voice notes recorded from the patient or other onsite operator of electronic stethoscope 10. Display screen 33 may provide questions for more in depth background information and voice notes associated with each question may be recorded. Some embodiments of electronic stethoscope 10 record the voice notes via sound transducer 42 in head 22 and programmable chip 36 may recognize simple voice commands to navigate and record voice notes. Other embodiments may have additional microphones on central circuit board 30 or elsewhere in electronic stethoscope 10. Accessories such as external microphones connected at port 32 may also be used, including head sets with microphones. Voice notes from a patient or onsite interviewer provide a more complete initial history when an audio file is to be reviewed by a remote practitioner.

Electronic stethoscope 10 is capable of real-time analysis of a measurement, or processing a measurement to be stored, including remotely such as in Cloud applications. When electronic stethoscope 10 is supplying real time analysis, programmable chip 36 drives display 33 to show the appropriate information. For heart measurements, display 33 may show a graph of the heart beat and display the heart rate. For lung measurements, display 33 may show a frequency spectrum analysis of the lung sounds. With real-time application, electronic stethoscope 10 can transmit audio to a receiver such as a Bluetooth headset or other similar audio devices.

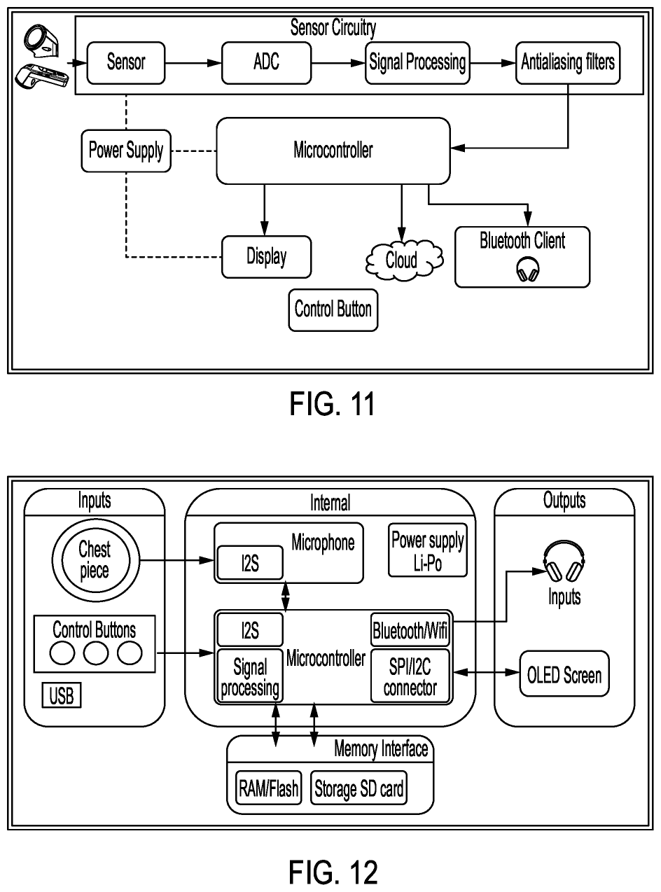

FIG. 11 is a flow chart of recording, signal processing, and communication of a measurement. A sound measurement of a body sound is recorded and converted from an analog form to digital form, and digital signal processing is then applied to the digital signal. The processed signal is then transmitted to a central microcontroller, or programmable chip, where it may be communicated to the onboard display or to external devices through any available transmission means. The external devices receiving transmission from electronic stethoscope 10 may also include cloud servers.

FIG. 12 shows interaction between elements of the system. Control buttons 34 on electronic stethoscope 10 allow a user to select settings for the operation of electronic microscope 10 via inputs into microcontroller, or programmable chip, 36. Microphone, or sound sensor, 41 associated with chest piece 24 in electronic stethoscope 10 records a body sound. Sound sensor 41 converts the signal from analog to digital and filters and conditions the signal before transmitting it to programmable chip 36. Programmable chip 36 performs analysis of the recorded signal by selection of appropriate baseline and comparing the current measurement to the baseline. Programmable chip 36 communicates the results to display screen 33 and external devices. Programmable chip 36 may also store the result in onboard memory within itself or another onboard chip 38 as shown in FIG. 5.

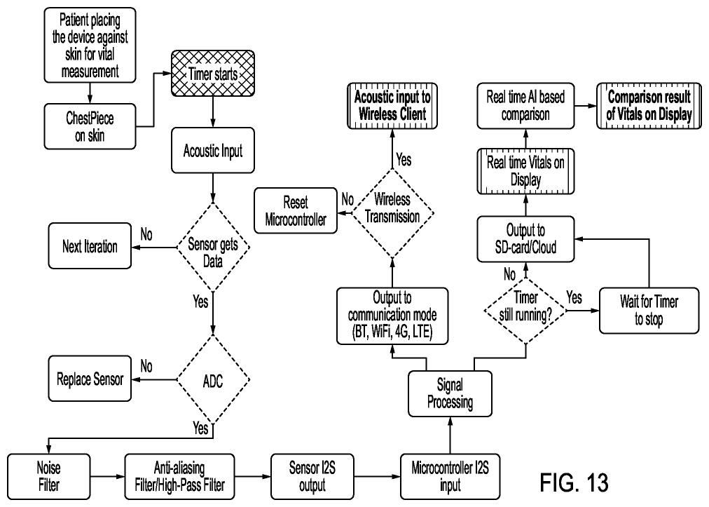

FIG. 13 shows the timed recording of a measurement through signal processing to automated analysis and comparison of the measurement. Upon confirmation of placement of electronic stethoscope 10 on a body by a contact sensor, sound sensor sound 41 begins a recording of body sounds for preset duration of time. This recording is converted from analog to digital and processed in real time to be transmitted to programmable chip 36 which converts the signal for communication to other devices. Upon completion of a timed recording, programmable chip 36 analyzes and compares the recording to an appropriate baseline selected from sampled data. The baseline is selected using artificial intelligence programming in programmable chip 36. The data and programming for analysis may be transmitted to programmable chip 36 by a port or by wireless communication.

Programmable chip 36 may do contrastive analysis between a current measurement and a baseline measurement. The baseline measurement may be a previous measurement or set of previous measurements of a patient, or the baseline measurement may be developed from a database of measurements of a larger population. When, the comparison is to a database, artificial intelligence (AI) tools may be employed to select the appropriate baseline.

In an initial step, an appropriate data set is chosen on a geographic basis. Differing geographic regions have different health profiles among the populations. These data sets are provided by health authorities, health counsels, etc. and consist of samples, or entries, for individuals with each entry having multiple pieces of information pertaining to the respective individual of that entry. Among the pieces of information are at least one target piece of interest for the sake of comparison, such as a heart rate. Once a data set from the appropriate geographic region is selected, Bootstrapping and Random Forest techniques are applied to the data set.

The selected data set is assumed to be an accurate representation of the relevant population and Bootstrapping is used to develop additional data sets from the data set of actual samples, or entries. A size is selected for the Bootstrapped data sets to be equal to or less than the sample data set size. Samples, or entries, are randomly selected from the original sample data set and added to the Bootstrapped data set in process until the Bootstrapped data set has the desired number of entries. The samples are chosen using a random number generator. Once the Bootstrapped data set has the correct number of copied samples, it is complete and ready for analysis or processing. Other Bootstrapped data sets may then be constructed from the original sample data set.

After multiple Bootstrapped data sets are constructed from the original sample data set, the Random Forest algorithm is applied to the datasets. The Bootstrapped data and the Random Forest algorithms are applied to determine the target value for a health parameter. In at least one embodiment, the health parameter is heartrate. Other health parameters may also be compared to a data set for a population.

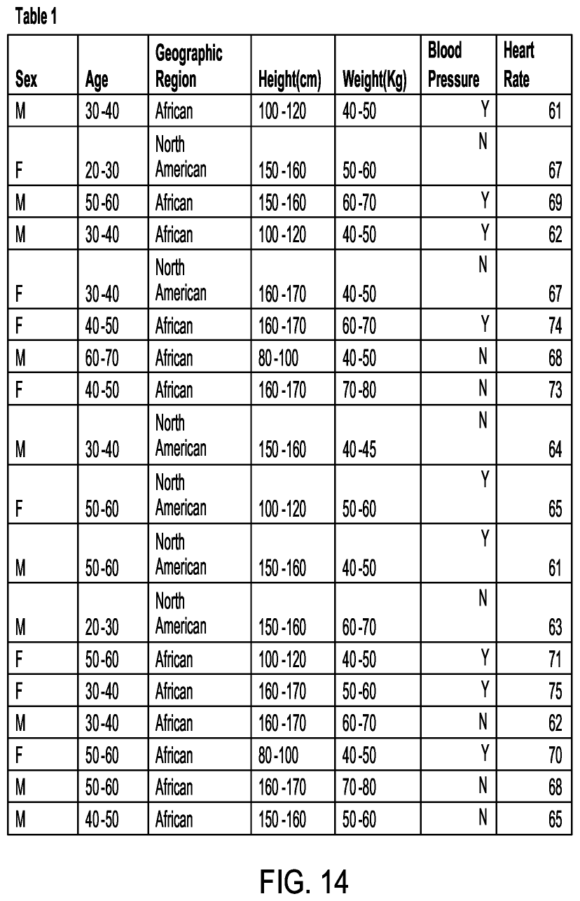

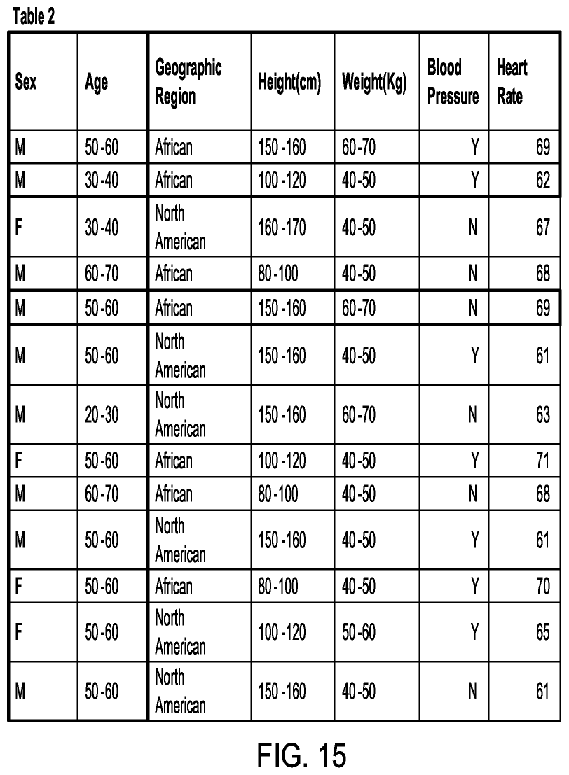

FIG. 14 is an example of a table, Table 1, containing a sampled data set. Each entry, or sample, represents an individual and contains multiple pieces of information about the individual. FIG. 15 is an example of a table, Table 2, containing a Bootstrap data set constructed from Table 1 of FIG. 14. As may be seen in these tables, several different pieces of information are recorded for each individual. In at least one embodiment, Geographic Region is used as an initial screen for the data. This means that when an individual's value of a health measurement is compared to a target value, the target value will be derived from samples from within the same geographic region.

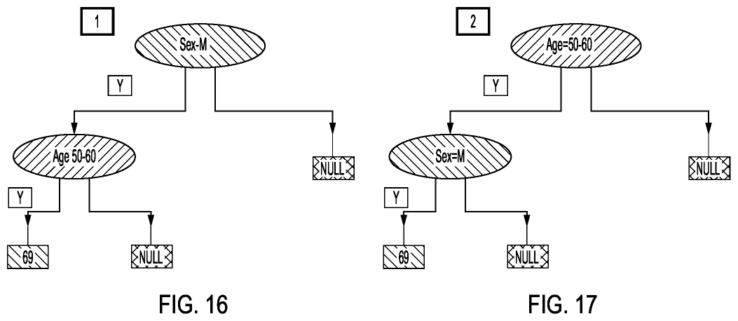

With the data segregated by geographic regions, the depth of the decision trees is selected as by how many variables will be used and therefore how many levels of decisions will be incorporated into the trees. All possible combinations of that number of variables, including the order of the variables, will be applied to bootstrapped data record and a decision tree created for each combination. This establishes the Random Forest to be used for data analysis. FIGS. 16 and 17 show two decision trees created over two variables in reverse order. Those decision trees were created from the first record of the Table 2, in FIG. 15.

With the Random Forest established, every decision tree is traversed using information from the current patient's profile. The value that the decision trees output the most total times is selected as the target value for that health parameter based on the patient's information. The measurement taken by electronic stethoscope 10 is compared to the target value selected by the Random Forest algorithm to evaluate the patient's status. For example, one common health metric is the heartrate of an individual. Based on an individual's information, the electronic stethoscope selects the appropriate baseline, or target heartrate, for comparison between the current patient and data. In one method of comparison, the percentage deviation of the current patient's heart rate from the target heartrate selected by the electronic stethoscope gives an indication of the patient's health. If the percentage deviation, high or low, is outside of set acceptable percentages, the patient is regarded as having a problematic heartrate calling for further examination. Once the patient has a record established in the system, the patient's heartrate may be compared to the patient's own history as well.

The wireless communication capabilities of electronic stethoscope 10 allow a patient's profile information as well as the patient's current measurements to be uploaded to the Cloud and stored for later review and consultation. Electronic stethoscope 10 can also retrieve previous patient records from the Cloud. This allows for the patient's metrics to be compared to his own baseline as well as a baseline selected based on patient data.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the invention be regarded as including such equivalent constructions.

We claim:

1. An electronic stethoscope for recording sounds produced from within a body, the electronic stethoscope comprising:
a housing;
a diaphragm at a surface of the housing;
a chest piece behind the diaphragm, the chest piece having an aperture through it;
a sound sensor within the housing positioned to receive sound from the aperture in the chest piece;
a programmable processor capable of executing machine readable instructions;
a display screen;
a battery;
user controls; and,
a contact sensor proximal to the diaphragm; wherein,
the programmable processor monitors the contact sensor for a contact signal indicating that the diaphragm is in position and the programmable processor operates a timer to control the duration of a recording from the sound sensor.

2. The electronic stethoscope of claim 1, wherein:
the programmable processor only continues the recording from the sound sensor while the contact sensor sends a contact signal.

3. The electronic stethoscope of claim 1, wherein:
the sound sensor comprises a microelectromechanical system and accompanying processor chip.

4. The electronic stethoscope of claim 1, wherein:
the contact sensor senses the force of contact.

5. The electronic stethoscope of claim 4, wherein:
the programmable processor requires that a minimum force be maintained at the contact sensor for the duration of the recording.

6. The electronic stethoscope of claim 1, wherein:
the contact sensor is a force sensitive resistor.

7. The electronic stethoscope of claim 1, wherein;
the programmable processor analyzes the signal from the contact sensor to determine whether the diaphragm is stationary on the body and records the signal from the sound sensor only when the programmable stethoscope determines that the diaphragm is stationary.

8. The electronic stethoscope of claim 1, further comprising;
a microphone for recording ambient sounds; wherein,
a signal from the microphone resulting from the ambient sounds is inverted and processed with the signal from the sound sensor to remove the effects of ambient sounds on the signal from the sound sensor.

9. The electronic stethoscope of claim 1, wherein:
the stethoscope compares selected metrics from the recording to respective baselines, the stethoscope selecting the respective baselines by applying the Bootstrap algorithm to a sampled dataset to create additional datasets and then applying the Random Forest algorithm to the additional datasets to select the respective baselines.

10. The electronic stethoscope of claim 1, wherein:
the timer is remotely adjustable.

11. A system, comprising:
the electronic stethoscope of claim 1 and external elements in communication with the electronic stethoscope.

12. The system of claim 11, wherein:
the external elements comprise at least one of the following;
a) cloud servers, or
b) a Bluetooth client.

13. An electronic stethoscope for recording sounds produced from within a body, the electronic stethoscope comprising:
a housing;
a diaphragm on the housing;
a sound sensor within the housing positioned to receive sound from the diaphragm;
a programmable processor capable of executing machine readable instructions;
a display screen;
user controls; wherein,
the stethoscope makes a recording of sounds and compares selected metrics from the recording to respective baselines, the stethoscope selecting the respective baselines by applying the Bootstrap algorithm to a sampled dataset to create additional datasets and then applying the Random Forest algorithm to the additional datasets to select the respective baselines.

14. The electronic stethoscope of claim 13, further comprising:
a contact sensor proximal to the diaphragm; wherein,
the programmable processor monitors the contact sensor for a contact signal indicating that the diaphragm is in position and the programmable processor operates a timer to control the duration of a recording from the sound sensor.

15. The electronic stethoscope of claim 14, wherein:
the programmable processor only continues the recording from the sound sensor while the contact sensor sends a contact signal.

16. The electronic stethoscope of claim 14, wherein:
the contact sensor senses the force of contact.

17. The electronic stethoscope of claim 16, wherein:
the programmable processor requires that a minimum force be maintained at the contact sensor for the duration of the recording.

18. The electronic stethoscope of claim 14, wherein;
the programmable processor analyzes the signal from the contact sensor to determine whether the diaphragm is stationary on the body and records the signal from the sound sensor only when the programmable stethoscope determines that the diaphragm is stationary.

19. The electronic stethoscope of claim 13, further comprising;
a microphone for recording ambient sounds; wherein,
a signal from the microphone resulting from the ambient sounds is inverted and processed with the signal from the sound sensor to remove the effects of ambient sounds on the signal from the sound sensor.

20. A system, comprising:
the electronic stethoscope of claim 13 and external elements in communication with the electronic stethoscope, wherein:
the external elements comprise at least one of the following;
a) cloud servers, or
b) a Bluetooth client.
